(19) United States
(12) Patent Application Publication
(10) Pub. No.: US 2004/0156797 A1
(43) Pub. Date: Aug. 12, 2004

(54) De' SaLa

(76) Inventor: Ghada Haddad, Houston, TX (US)

Correspondence Address: Ghada Haddad, 3739 Cypress Hills, Spring, TX, 77388, US

(21) Appl. No.: 10/263,238

(22) Filed: Feb. 10, 2003

(51) Int. Cl. A61K 7/42

(52) U.S. Cl. 424/59

(57) ABSTRACT

Our product helps to:

Increase enzyme activity that protects against damaging changes to cells.

It helps healing the signs of infection (hotness, redness, tenderness)

It helps the process of replacing the damaged cells, visibly noticed on cold sores on lips, and especially inside the mouth.

Fruit's juice Products experimentally have been proven to be nutritionally supports healthy skin.

For thousands of years the Middle Eastern people used this special product for its exceptional skin healing properties.

It may help to give you smooth skin, remove scar tissue, signs of skin inflammation and replace the damage cells.

This statement has not been evaluated by the FDA. The product is not intended to diagnose, treat, cure or prevent any disease.

The Ingredients:

Natural Ellagic Acid (from fruits Extracts), Sage, Jirofler, Chamomile

### THE INVENTOR'S BACKGROUND

She grown up in Middle East, licensed as Register Nurse from UK and Wales, licensed as an Esthetician from Texas board USA and the owner of Maran Mediterranean day spa.

She is an expert in Facial, Acne treatments . . . etc

### HOW TO PREPARE THE PRODUCT

Add all the fruits juice together, which are rich in Natural Ellagic Acid, mix it well, Concentrated by boiling, add Sage, Jirofler and Chamomile.

Let it cool and then filter it. The concentrate will show a great affect on skin, especially for the Acne.

How to Use the Product:

1-Clean the skin thoroughly before applying the product.

2-Apply a thin layer directly on the affected areas 2 to 3 times daily or till the acne dry.

Warning:

Avoid contact with eyes.

Test the product skin allergy before using it: apply the product on a small area of your skin and wait for 24 hours.

If you have a sensitive skin, don't use that product.

Keep it out of the reach of the children. In case of accidental ingestion, drink lot of water and seek professional assistance.

Active Ingredient:

Natural Ellagic Acid from fruits extracts.

What I claimed as my invention is:

1. A product for helping skin pimples and cold sores, exfoliates, helps aging spots on hands, helps dark areas on skin, helps soften, smooth and sooth the skin.
This product boots sun protection topically and internally.
